(19) United States
(12) Patent Application Publication
(10) Pub. No.: US 2008/0207667 A1
(43) Pub. Date: Aug. 28, 2008

(54) Use of nalbuphine and related compounds to treat symptoms of respiratory problems

(76) Inventor: Robert W. Rhame, Eutawville, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 11/710,383

(22) Filed: Feb. 23, 2007

(51) Int. Cl. A61K 31/485

(52) U.S. Cl. 514/282

(57) ABSTRACT

The present invention relates to treatment of respiratory diseases. More specifically, the present invention relates to treatment of respiratory diseases in humans and lower animals with nalbuphine.

### BACKGROUND OF THE INVENTION

The present invention relates generally to the treatment of respiratory diseases. More specifically, the invention relates to the use of nalbuphine and related compounds to treat respiratory diseases.

Nalbuphine is a synthetic opoid used commercially as an analgesic under a variety of trade names, including NUBAIN™. More specifically, nalbuphine is a synthetic narcotic agonist-antagonist analgesic of the phenanthrene series. It is chemically related to both the widely used narcotic antagonist nalaxone, and the potent narcotic analgesic, oxymorphone,

Nalbuphine is known as having an analgesic potency essentially equivalent to that of morphine on a milligram basis. When used for its analgesic properties, the onset of action typically occurs within two to three minutes after intravenous administration, and in less than fifteen minutes following subcutaneous or intramuscular injection. The narcotic antagonist activity of nalbuphine is one-fourth as potent as nalorphine and ten times that of pentazocine.

Nalbuphine is indicated for the relief of moderate to severe pain. It can also be used as a supplement to balanced anesthesia, for preoperative and postoperative analgesia, and for obstetrical analgesia during labor and delivery. Although nalbuphine possesses narcotic antagonist activity, there is evidence that in nondependent patients, it will not antagonize a narcotic analgesic administered just before, concurrently, or just after an injection.

Common side effects of nalbuphine include sedation, feeling sweaty/clammy, nausea/vomiting, dizziness/vertigo, dry mouth, and headache. Other listed side effects include central nervous system effects (i.e., nervousness, depression, restlessness, crying, euphoria, floating, hostility, unusual dreams, confusion, faintness, hallucinations, dysphoria, feeling of heaviness, numbness, tingling, and unreality. Respiratory side effects include respiratory depression, dyspnea, and asthma.

Romagnoli and Keats reported, in Clin. Pharmacol. Ther. 1980, April; 27(4):478-85, that nalbuphine has a respiratory depressant capacity similar to that of morphine. The authors recognized, however, that nalbuphine possesses a ceiling effect for respiratory depression. Stated differently, the dose effect curve for respiratory depression by nalbuphine was flatter than that of morphine, and maximum respiratory depression occurred after 30 mg of nalbuphine per 70 kg body weight was administered. Additionally, doses in excess of 30 mg/70 kg failed to increase respiratory depression beyond that induced by morphine at 20 mg/70 kg. This ceiling effect demonstrates a unique safety factor for nalbuphine among analgesics.

Additional research has shown that when nalbuphine is administered following or concurrent with mu agonist opoid analgesics (e.g., morphine, oxymorphone, fentanyl), nalbuphine may partially reverse or block opoid-induced respiratory depression from the mu agonist analgesic (www.drugs.com/PDR/Nubain lniection.html).

As previously stated, nalbuphine is recognized as inducing respiratory depression. Published drug precautions stated that nalbuphine should be administered with caution at low does to patients with impaired respiration (e.g., from other medication, uremia, bronchial asthma, severe infection, cyanosis, or respiratory obstructions) (www.drugs.com).

U.S. Pat. No. 6,680,067, to Hu, et al., is directed to controlled-release pharmaceutical preparation containing nalbuphine and a process for preparing the preparation. Hu recognizes nalbuphine as being useful in the treatment of pain associated with cardiac, pulmonary, abdominal, osteopathic or obstetrical surgery, severe burn injury, and the terminal stage of cancer. Hu's controlled release preparation is directed to the treatment of pain over an extended period of time. Hu does not recognize the use of nalbuphine to treat the diseases themselves, only the pain associated with those diseases.

Nalbuphine has not been recognized as being capable of reversing diseases affecting the respiratory system such as asthma, chronic obstructive pulmonary disease, cough, and cough with pruritis. Previous research, as stated above, indicates that nalbuphine is contraindicated for treatment of diseases affecting the respiratory system.

It would be desirable, therefore, to develop nalbuphine as a treatment for diseases affecting the respiratory system due to the low incidence of side effects reported from the use of nalbuphine and the ready availability of nalbuphine.

### SUMMARY OF THE INVENTION

Briefly, therefore, the present invention is directed to a method for treating respiratory diseases. The method includes administering to a human or a lower animal safe and effective amounts of nalbuphine.

In another aspect, the invention is directed to a combination including a container. The container includes a means for topical application selected from the group consisting of dropper means, spray means, and inhalation mist means. The container includes therein a composition for treatment of symptoms associated with respiratory diseases, in the form of an aqueous solution comprising a safe and effective amount of one or both of nalbuphine or a pharmaceutically acceptable salt thereof.

In yet another aspect, the invention is a composition including one or both of nalbuphine and a pharmaceutically acceptable salt thereof, and a safe and effective amount of another drug active selected from the group consisting of antihistamines, decongestants, expectorants, bronchodilators, antitussives, and combinations thereof.

Among the several advantages found to be achieved by the present invention, therefore, may be noted the provision of a novel method for the treatment of respiratory diseases. Additionally, the successful use of nalbuphine to treat respiratory diseases is unexpected due to the recognized side effects of nalbuphine, including respiratory depression.

### DETAILED DESCRIPTION OF SEVERAL EMBODIMENTS

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention cover such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In accordance with the present invention, it has been discovered that nalbuphine and related compounds may be used in the successful treatment of respiratory diseases and their symptoms. For example, the present invention relates to the treatment of asthma, cough, cough with pruirtis, chronic obstructive pulmonary disease, dyspnea, the symptoms of the listed diseases, and other respiratory diseases and their symptoms.

In one aspect, the invention is a method of treatment of respiratory diseases and their symptoms in humans and lower animals. The method includes administration to the human or lower animal safe and effective amounts of a composition including nalbuphine. As used herein, the term “safe and effective” will vary depending on the subject to whom the composition is being administered. For example, a safe and effective dose of nalbuphine for a human may be different than a safe and effective dose for a lower animal. Those having ordinary skill in the art will recognize that the definition of “safe and effective” will vary accordingly.

For ease of reference, the present invention will be described with reference to administration to humans. It will be understood, however, that such descriptions are not limited to administration to humans, but will also include administration to other animals, such as mammals, unless explicitly stated otherwise.

The present method includes administering the composition to the subject by administration means known in the art. Administration means contemplated as useful include one or more of topically, buccally, intranasally, orally, intravenously, intramuscularly, sublingually, and subcutaneously. Other administration means known in the art are also contemplated as useful in accordance with the present invention.

In some embodiments, it may be useful to include nalbuphine as a salt, more specifically as nalbuphine hydrochloride.

The composition administered in accordance with the present invention may also include one or more of antihistamines, decongestants, expectorants, bronchodilators, beta-2-agonists, opoid agonists, opoid antagonists, antitussives, excipients, steroidal anti-inflammatory drug agents, non-steroidal anti-inflammatory drug agents, and anticholinergic drug agents.

In some embodiments, the composition may be an aqueous composition. The composition may also be nebulized or aerosolized.

The subject invention involves the use of a safe and effective amount of nalbuphine for the treatment of respiratory diseases and the symptoms associated with respiratory diseases, such as colds, flu, allergic and vasomotor rhinitis, asthma, and bronchitis, of humans and lower animals, especially humans. Treatment of both upper respiratory symptoms, such as nasal congestion runny nose, sneezing, and post-nasal drip, as well as lower respiratory symptoms, such as bronchoconstriction and cough are contemplated in accordance with the present invention.

An exemplary method of administering the nalbuphine is topical, intranasal administration, e.g., with nose drops, nasal spray, or nasal mist inhalation. Other exemplary methods of administration include one or more of topical, bronchial administration by inhalation of vapor and/or mist or powder, orally, intravenously, intramuscularly, and subcutaneously.

Other ingredients which may be incorporated in the present invention include safe and effective amounts of preservatives, e.g., benzalkonium chloride, thimerosal, phenylmercuric acetate; and acidulants, e.g., acetic acid, citric acid, lactic acid, and tartaric acid. The present invention may also include safe and effective amounts of isotonicity agents, e.g., salts, such as sodium chloride, and more preferably non-electrolyte isotonicity agents such as sorbitol, mannitol, and lower molecular weight polyethylene glycol.

A particularly preferred ingredient of the compositions of the subject invention is a safe and effective amount of a solubilizing agent. Nalbuphine and its salts are sparingly soluble in water. A suitable solubilizing agent increases the solubility of nalbuphine and/or its salts in the aqueous compositions. Such solubilizing agents can also provide isotonicity for the aqueous compositions. Preferred solubilizing agents are modified cyclodextrins, preferably hydroxy-C.sub.1-C.sub.6 alkyl derivatives, especially hydroxypropyl derivatives. A particularly preferred solubilizing agent is 2-hydroxypropyl-.beta.-cyclodextrin. Solubilizing agents are preferably present in the compositions of the subject invention at a concentration of from about 0.1% to about 10%, more preferably from about 0.5% to about 5%.

The compositions of the subject invention also may comprise safe and effective amounts of one or more other active drug agents useful for treating the respiratory diseases of interest. Such other active drug agents and typical amounts dosed are disclosed in Physician's Desk Reference, 44th Edition (1990), E. R. Barnhardt, publisher, and Physician's Desk Reference for Nonprescription Drugs, 11th Edition (1990), E. R. Barnhardt, publisher, both of which are incorporated herein by reference.

The topical nasal compositions of the subject invention may include one or more of the following such other active drug agents: antihistamines, e.g., chlorpheniramine maleate, pyrilamine maleate, diphenhydramine hydrochloride, promethazine hydrochloride, doxylamine succinate, terfenadine, astemizole; decongestants, e.g., pseudoephedrine hydrochloride, phenyl propanolamine hydrochloride, phenylephrine hydrochloride, oxymetazoline hydrochloride, xylometazoline hydrochloride; steroidal anti-inflammatories, e.g., beclomethasone dipropionate, flunisolide; mast cell stabilizers, e.g., cromolyn sodium, nedocromil; anticholinergics, e.g., ipratropium bromide.

The topical bronchial compositions of the subject invention may include one or more of the following such other active drug agents: antitussives, e.g., dextromethorphan base or hydrobromide; bronchodilators, e.g., theophylline, metaproterenol, albuterol; steroidal anti-inflammatories, e.g., beclomethasone; mast cell stabilizers, e.g., cromolyn sodium.

Oral compositions of the subject invention may include one or more of the following such other active drug agents: antihistamines, e.g., chlorpheniramine maleate, pyrilamine maleate, diphenhydramine hydrochloride, promethazine hydrochloride, doxylamine succinate, terfenadine, astemizole; decongestants, e.g., pseudoephedrine hydrochloride, phenylpropanolamine hydrochloride, phenylephrine hydrochloride; antitussives, e.g., dextromethorphan base or hydrobromide; nonsteroidal anti-inflammatories, e.g., aspirin, acetominophen, ibuprofen, naproxen; expectorants, e.g., guaifenesin; bronchodilators, e.g., theophylline, metaproterenol, albuterol; and antibiotics.

Another aspect of the subject invention is a combination of a composition comprising an above compound in a container comprising a means for topical application of the composition to the eyes, nasal passages and sinuses, or bronchial passages and lungs. Preferred containers useful in such combinations include those comprising dropper means, spray means, or inhalation mist or powder means.

Containers comprising dropper means are useful for applying, as a liquid, either eye drops or nose drops topically to the eye or nasal passages, respectively. Such containers are well-known and commonly have such dropper means attached permanently or removably to the body of the container so that drops can be administered by inverting the container and/or by squeezing the container (the container being flexible). Another well-known dropper means is attached to a closure for the container and comprises a tube with a small hole in one end, the other end being open and attached to a flexible (e.g., rubber) bulb.

Containers comprising spray means are useful for applying a spray of liquid droplets topically directly to nasal passages. Well-known examples of such containers are flexible plastic containers having a spray nozzle fixedly attached thereto, the spray nozzle being designed for insertion into the nasal opening. When the container is squeezed, solution in the container is forced through the nozzle and emerges as a spray of droplets. Other well-known containers with spray means, e.g. pump sprays or aerosol sprays, can also be used in a similar manner.

Containers comprising inhalation mist means are useful for applying a fine mist or powder topically to nasal passages and/or bronchial passages and lungs. Such inhalers provide a fine mist or powder which can be inhaled either through the nose or the mouth, depending on the design of the inhaler. Inhalers designed for providing a mist or powder to be inhaled through the nose are useful for topical administration of compositions to nasal passages and/or bronchial passages and lungs. Inhalers designed for providing a mist or powder to be inhaled through the mouth are useful for topical administration of compositions to bronchial passages and lungs. Various containers having inhalation mist or powder means as a part of or fixedly attached to the containers are well-known, e.g., squeeze containers, pump containers, and aerosols.

In the present method, a subject in need of prevention or treatment of a respiratory disorder is treated with an amount of nalbuphine, where the amount of the nalbuphine provides a dosage or amount of the combination that is sufficient to constitute a respiratory disorder treatment or prevention effective amount.

As used herein, an “effective amount” means the dose or effective amount to be administered to a patient and the frequency of administration to the subject which is readily determined by one or ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the patient to be treated, and other relevant circumstances.

The phrase “therapeutically-effective” indicates the capability of an agent to prevent, or improve the severity of, the disorder, while avoiding adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

In the present method, the amount of nalbuphine that is used in the novel method of treatment preferably ranges from about 0.1 to about 1.00 milligrams per day per kilogram of body weight of the subject (mg/day·kg), more preferably from about 0.285 to about 0.57 mg/day kg.

The amount of nalbuphine that is used in the subject method may be an amount that is sufficient to constitute a respiratory disorder treatment or prevention effective amount.

When the Cox-2 selective inhibitor comprises rofecoxib, it is preferred that the amount used is within a range of from about 0.15 to about 1.0 mg/day·kg, and even more preferably from about 0.18 to about 0.4 mg/day·kg.

The nalbuphine can be supplied in the form of a novel therapeutic composition that is believed to be within the scope of the present invention.

When the nalbuphine is supplied along with a pharmaceutically acceptable carrier, a pharmaceutical composition is formed. A pharmaceutical composition of the present invention is directed to a composition suitable for the prevention or treatment of respiratory disorders. The pharmaceutical composition comprises at least a pharmaceutically acceptable carrier and nalbuphine. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in present invention are the isomeric forms and tautomers and the pharmaceutically-acceptable salts of nalbuphine. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

As used herein, the term “respiratory disorders” is meant to include, without limitation, each of the symptoms or diseases that is mentioned above.

The present method includes the treatment and/or prevention of respiratory disorders in a subject, where the method comprises treating the subject having or susceptible to the disorder with a therapeutically-effective amount of nalbuphine, such as described in this specification.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of respiratory disorders associated with, but not limited to, any of the diseases or disorders described above. Besides being useful for human treatment, these combinations are also useful for treatment of mammals, including horses, dogs, cats, rats, mice, sheep, pigs, etc.

The term “subject” for purposes of treatment includes any human or animal subject who is in need of the prevention of, or who has respiratory disorders. The subject is typically a human subject.

For methods of prevention, the subject is any human or animal subject, and preferably is a subject that is in need of prevention and/or treatment of pain, inflammation and/or an inflammation-associated disorder. The subject may be a human subject who is at risk respiratory disorders, such as those described above. The subject may be at risk due to genetic predisposition, sedentary lifestyle, diet, exposure to disorder-causing agents, exposure to pathogenic agents and the like.

The pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrase “therapeutically-effective” and “effective for the treatment, prevention, or inhibition”, are is intended to qualify the amount of each agent for use in the combination therapy which will achieve the goal of improvement in inflammation severity and the frequency of incidence over treatment of each agent by itself, while avoiding adverse side effects typically associated with alternative therapies.

In particular, the nalbuphine compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables;

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems include sprays, capsules, tablets, and gelatin capsules, for example.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### EXAMPLE 1

This example illustrates the use of nalbuphine to treat a subject suffering from a respiratory disorder. The subject was a 51 year old female former smoker. Spirometry measurements were taken pre and post treatment with nebulized nalbuphine. The following measurables were observed:

| | Pre-Nalbuphine | | |
|---|---|---|---|
| | | | % of |
| | Predicted | Actual | Prediction |
| FVC (L) | 3.789 | 3.507 | 93 |
| FEV1 (L) | 3.029 | 2.892 | 95 |
| FEV1/FVC | 79 | 82 | 105 |
| (%) | | | |
| FEF (25–75%) | 3.883 | 3.667 | 94 |
| (L/S) | | | |
| PEF (L/S) | 6.453 | 6.391 | 99 |
| Time (sec) | | 4.9 | |

| | Post-Nalbuphine: Treatment #1 | | |
|---|---|---|---|
| | | % of | % |
| | Actual | Prediction | Change |
| FVC (L) | 5.035 | 133 | 44.0 |
| FEV1 (L) | 4.223 | 139 | 46 |
| FEV1/FVC | 84 | 106 | 2 |
| (%) | | | |
| FEF (25–75%) | 5.043 | 130 | 37.50 |
| (L/S) | | | |
| PEF (L/S) | 5.98 | 93 | −6.4 |
| Time (sec) | 4.117 | | |

| | Post-Nalbuphine: Treatment #2 | | |
|---|---|---|---|
| | | % of | % |
| | Actual | Prediction | Change |
| FVC (L) | 4.827 | 127 | 38 |
| FEV1 (L) | 4.259 | 134 | 40 |
| FEV1/FVC | 84 | 107 | 2 |
| (%) | | | |
| FEF (25–75%) | 5.327 | 137 | 45 |
| (L/S) | | | |
| PEF (L/S) | 7.297 | 113 | 14 |
| Time (sec) | 7.117 | | |

| | Post-Nalbuphine: Treatment #3 | | |
|---|---|---|---|
| | | % of | % |
| | Actual | Prediction | Change |
| FVC (L) | 4.733 | 125 | 35 |
| FEV1 (L) | 4.11 | 136 | 42 |
| FEV1/FVC | 87 | 110 | 6 |
| (%) | | | |
| FEF (25–75%) | 4.998 | 129 | 36 |
| (L/S) | | | |
| PEF (L/S) | 7.225 | 112 | 13 |
| Time (sec) | 4.517 | | |

The measurables of Example 1 demonstrate the effectiveness of the present methods. As can be seen, by the forty-four percent increase in Forced Vital Capacity (FVC) and the forty six percent increase in the Forced Expiratory Volume (FEV1) in one second, there is marked improvement in lung capacity and airflow after treatment with nalbuphine.

### EXAMPLE 2

In this example, an eighty year old female asthmatic taking advair diskus for treatment of asthma was treated with nalbuphine according to the present method. Spirometry measurements were taken pre and post treatment with nebulized nalbuphine. The following measurables were observed:

| | Pre-Nalbuphine | | |
|---|---|---|---|
| | | | % of |
| | Predicted | Actual | Prediction |
| FVC (L) | 2.158 | 0.856 | 40 |
| FEV1 (L) | 1.576 | 0.375 | 24 |
| Time (sec) | | 4.933 | |

| | Post-Nalbuphine | | |
|---|---|---|---|
| | Actual | % of Prediction | % Change |
| FVC (L) | 1.155 | 54 | 35 |
| FEV1 (L) | 0.555 | 35 | 48 |
| Time (sec) | 6.967 | | |

The measurables of Example 2 demonstrate the effectiveness of the present methods. As can be seen, there is a dramatic increase in FVC and FEV1, again demonstrating marked improvement in lung capacity and airflow after treatment with nalbuphine.

### EXAMPLE 3

In this example, a 70 year old male smoker currently being treated with combivent and spiriva was treated with nalbuphine according to the methods of the present invention. Spirometry measurements were taken pre and post treatment with nebulized nalbuphine. The following measurables were observed:

| | Pre-Nalbuphine | | |
|---|---|---|---|
| | | | % of |
| | Predicted | Actual | Prediction |
| FVC (L) | 4.215 | 2.453 | 58 |
| FEV1 (L) | 3.253 | 1.968 | 61 |
| Time (sec) | | 3.617 | |

| | Post-Nalbuphine | | |
|---|---|---|---|
| | Actual | % of Prediction | % Change |
| FVC (L) | 3.285 | 78 | 34 |
| FEV1 (L) | 2.745 | 84 | 39 |
| Time (sec) | 5.25 | | |

The measurables of Example 3 demonstrate the effectiveness of the present methods. As can be seen, treatment with nalbuphine produced a marked improvement in lung capacity and airflow as evidenced by the change in the FVC and FEV1 values

Each example illustrates the effectiveness of the present methods in treating respiratory disorders. The examples are provided for the purposes of illustration only and should not be construed in a limiting manner.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties.

The discussion of the references herein is intended merely to summarize the assertions made by their authors and no admission is made that any reference constitutes prior art. Applicants reserve the right to challenge the accuracy and pertinency of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part.

What is claimed is:

1. A method of treatment of symptoms associated with diseases affecting the respiratory or pulmonary system of humans and lower animals, comprising administration to the human or lower animal safe and effective amounts of a composition including nalbuphine.

2. The method according to claim 1, wherein the symptoms associated with diseases affecting the respiratory system include one or more of asthma, cough, bronchoconstriction, cough with pruritis, chronic obstructive pulmonary disease, dyspnea, pulmonary hypertension, or pulmonary symptoms, and distress associated with cardiac disease or failure or cancerous processes involving the respiratory system.

3. The method according to claim 1, wherein the composition is administered by methods including one or more of topically, intranasally, orally, intravenously, intramuscularly, via the buccal mucosa, and subcutaneously.

4. The method according to claim 1, wherein the composition includes a salt of nalbuphine.

5. The method according to claim 4, wherein the salt of nalbuphine is nalbuphine hydrochloride.

6. The method according to claim 1, wherein the composition further includes one or more of antihistamines, decongestants, expectorants, bronchodilators, beta-2-agonists, opoid agonists, opoid antagonists, and antitussives.

7. The method according to claim 1, further comprising an excipient.

8. The method according to claim 1, further comprising one or both of a steroidal or nonsteroidal anti-inflammatory drug agent.

9. The method according to claim 1, further comprising an anticholinergic drug agent.

10. The method according to claim 1, wherein the composition is in the form of an aqueous solution.

11. The method according to claim 1, wherein the composition is nebulized.

12. The method according to claim 1, wherein the composition is aerosolized.

13. A combination comprising:
a container comprising a means for topical application selected from the group consisting of dropper means, spray means, and inhalation mist means, the container containing:
a composition for treatment of symptoms associated with respiratory diseases, in the form of an aqueous solution comprising a safe and effective amount of one or both of nalbuphine or a pharmaceutically acceptable salt thereof.

14. The combination according to claim 13, wherein the container further comprises a suitable excipient.

15. The combination according to claim 13, further comprising a drug agent selected from the group consisting of antihistamines, decongestants, expectorants, bronchodilators, beta-2-agonists, opoid agonists, opoid antagonists, and antitussives.

16. The combination according to claim 13, further comprising one or both of a steroidal and non-steroidal anti-inflammatory drug agent.

17. The combination according to claim 13, further comprising an anticholinergic drug agent.

18. A composition for treatment of symptoms associated with respiratory diseases, the composition comprising one or both of nalbuphine and a pharmaceutically acceptable salt thereof, and a safe and effective amount of another drug active selected from the group consisting of antihistamines, decongestants, expectorants, bronchodilators, beta-2-agonists, opoid agonists, opoid antagonists, antitussives, and combinations thereof.

19. The composition according to claim 18, wherein the composition is in the form of a tablet, an aqueous solution, or an inhalation mist.

20. The composition according to claim 18, further comprising a preservative.

21. The composition according to claim 18, further comprising a steroidal or non-steroidal anti-inflammatory drug agent.

22. The composition according to claim 18, further comprising an anticholinergic drug agent.
